(19) United States
(12) Patent Application Publication
(10) Pub. No.: US 2009/0004326 A1
(43) Pub. Date: Jan. 1, 2009

(54) Beverage Additive and Method of Making the Same

(76) Inventor: Randy Andrews, West Linn, OR (US)

Correspondence Address: PETER A. HAAS, ESQUIRE, 1929 SW 13TH AVENUE, PORTLAND, OR, 97201, US

(21) Appl. No.: 12/163,585

(22) Filed: Jun. 27, 2008

Related U.S. Application Data
(60) Provisional application No. 60/947,230, filed on Jun. 29, 2007.

(51) Int. Cl. A23F 5/20; A23L 1/30; A61K 36/28; A61K 36/48

(52) U.S. Cl. 426/2; 426/594

(57) ABSTRACT

In one embodiment, the present invention comprises a food additive for beverages, specifically suited as an additive for coffee or other coffee beverages that reduces the effects of the acidic beverage on a human's digestive system. In one embodiment, a form of DGL and other all-natural components reduce the effects of the acidity of coffee without negatively affecting the flavor. In other embodiments, the food additive includes nutritional supplements including vitamins and minerals and other ingredients to transform coffee into a functional beverage.

### PRIORITY CLAIM

The present application claims benefit under 35 USC Section 119(e) of U.S. Provisional Patent Application Ser. No. 60/947,230 filed on 29 Jun. 2007, the disclosure of which is hereby expressly incorporated herein by reference.

### BACKGROUND

The present invention relates to food additives, specifically acid reducing, reducing acidic-affects, and functional additives for beverages, namely coffee.

Coffee is consumed on a daily basis by millions of people worldwide. Other than enjoyment of the flavor, one of the only physical benefits of consuming coffee is as a stimulant that may make one feel more awake or alert. There is a potential, not adequately addressed in the prior art, to provide health benefits to coffee by incorporating an additive and, thus, transforming coffee into a functional beverage.

One, specific health disadvantage to coffee arises from its inherent acidity. The degree of any beverage's acidity or alkalinity, which can be measured as a potential of Hydrogen, can potentially be harmful. The term potential of hydrogen, represented by the letters “pH”, is measured on a scale of 0 to 14. The lower the pH number, the more acidic the solution and, conversely, the higher the pH value the more basic or alkaline the solution. A neutral solution that is neither acidic nor alkaline has a pH value of 7. Unfortunately for many beverage drinkers, certain beverages, including coffee, are restricted or eliminated from their diet because their bodies cannot safely digest and process such acidic beverages.

There is a natural and normal level of acidity in human's bodies. And, this normal range from about a saliva pH of about 7.0 to 7.5 is healthy and essential—for example, the stomach secretes acid that is essential in the digestive process and this acid helps in breaking down the food during digestion. A healthy body with a pH-balanced environment allows normal body function and promotes resistance to disease. Accordingly, a normal and healthy body maintains adequate alkaline reserves to meet emergency demands.

But, when there is an excess of acid certain problems arise. One such common health-related problem is acidity. This excess acid in the body forces the body to transfer minerals—primarily calcium and magnesium—from vital organs and bones to counteract the acidic condition and buffer (or neutralize) the acid and safely remove it from the body. Untreated, an acidic condition strains the body and can lead to severe and prolonged damage. Worse, such a condition may go undetected for years. In an overly acidic body the excess acid levels are countered by natural response systems that attempt to neutralize the overly acidic condition and return the body to a balanced condition. To do this, a person's body allocates alkaline reserves to attack the excess acidity. This, in turn, depletes the body's alkaline reserves leaving the body in a weakened condition. This acidic condition, also termed “over acidity”, dangerously weakens all body systems and commonly occurs in many adults throughout the world. Over acidity, left untreated, results in an unhealthy body having an internal environment conducive to disease.

Common ailments related to an acidic condition include:
1. Cardiovascular damage, including the constriction of blood vessels and the reduction of oxygen;
2. Weight gain, obesity and diabetes;
3. Bladder and kidney conditions, including kidney stones;
4. Immune deficiency;
5. Accelerated free radical damage, which contributes to cancerous mutations;
6. Premature aging;
7. Osteoporosis including weak, brittle bones, hip fractures, and bone spurs;
8. Joint pain, aching muscles and lactic acid buildup; and
9. Low energy, and chronic fatigue.

A recent seven-year study, conducted at the University of California, San Francisco on 9,000, women showed that those who have chronic acidosis are at greater risk for bone loss than those who have normal pH levels. Many hip fractures—prevalent among middle-aged women—are connected to high acidity. Other symptoms of an acidic condition include dyspepsia, heartburn, and the formation of ulcers (erosion of the lining of the stomach or intestines).

Consuming certain foods may lead to an acidic condition. For example, consumption of coffee, alcohol, highly spicy foodstuffs, non-vegetarian diets, and Non Steroidal Anti-Inflammatory Drugs (NSAID's) also predispose to gastric acidity.

The stomach, intestines, and digestive glands secrete hydrochloric acid and various enzymes, including pepsin that break down and digest food. The stomach must also be protected from the same acid and enzymes, or it too can be attacked by the gastric juices. The acid may enter the lower part of the Esophagus (Gastro-Esophageal Reflux), due to some weakness in the normal sphincter mechanism that prevents such reflux. This causes heartburn. It commonly occurs after specific beverages or meals.

Ulcers also occur as a result of over secretion of acid. This may happen when there is an imbalance between the digestive juices used by the stomach to break down food and the various factors that protect the lining of the stomach and duodenum (the part of the small intestine that adjoins the stomach).

A peptic ulcer is a raw area in the lining of the upper part of the small intestine (duodenal ulcer) or the stomach (gastric ulcer), whose protective mucosal lining has been eroded away by the gastric juices. Duodenal ulcers are three times more common than gastric ulcers. Hydrochloric acid, secreted in the stomach, is one of the factors in the development of ulcers, but is not solely responsible. Acid production in patients with duodenal ulcers tends to be higher than normal, while in those with stomach or gastric ulcers; it is usually normal or lower.

Excessively large amounts of acid secretion occur in certain situations, such as in a condition known as Zollinger-Ellison Syndrome, in which large amounts of secretion are stimulated by tumors located in the pancreas or duodenum.

Pepsin is an enzyme that breaks down proteins. Pepsin and hydrochloric acid cause damage to the stomach or duodenum if the stomach's protective system is altered or damaged. The mucous layer, which coats the stomach and duodenum, forms the first line of defense against acid and pepsin.

The body also secretes bicarbonate into the mucous layer, which neutralizes the acid. The defense system also consists of hormone-like substances known as prostaglandins, which help to keep the blood vessels in the stomach dilated, ensuring adequate blood flow. Lack of adequate blood flow to the stomach contributes to ulcers. Prostaglandins are also believed to stimulate bicarbonate and mucous production, which help protect the stomach. If any of these defense mechanisms are deficient, acid and pepsin can attack the stomach lining causing an ulcer.

Persons with an acidic condition typically must reduce their intake of certain foods and beverages as treatment. One typical beverage commonly eliminated from the diet of such persons is coffee. However, coffee one of the most popular beverages world-wide, is consumed at a volume of about a third of that of tap water in North America and Europe, and, in 2003, coffee was the world's sixth largest agricultural export in value. But, the elimination of coffee from one's diet is difficult for many people.

Coffee plays an important economic and social role: it serves a ritual function in modern society and many people simply crave its flavor. Recognizing that there is a huge demand for coffee, even within the population of people suffering from an acidic condition, certain attempts to negate the harmful effect of coffee are discussed in the prior art. For example, U.S. Pat. No. 5,849,346 to Hornack issued on 15 Dec. 1998 describes a beverage additive that neutralizes the acidity of a beverage to provide the body a proper balance of needed electrolytes. Therein, an aqueous solution of potassium hydroxide and a mixture of electrolyte ions including sodium, calcium, magnesium, chloride, bicarbonate, phosphate, and sulfate store in a dropper bottle so that the additive can be carried in a pocket or purse so it may be readily at hand to be added to a person's beverage. One limitation of this approach, however, includes that the aqueous additive alters the flavor of the beverage, especially when that beverage is coffee. Because it is the acidity of the coffee that provides the bright and dry taste that coffee drinkers enjoy. (Note: The term “coffee acidity” is easily misunderstood with the aforementioned acidic condition. Coffee acidity, which is not necessarily directly correlated to a particular pH value, is a coffee characteristic that plays a key role in the flavor and coffee taste. To avoid confusion with a person's acidic condition, this document hereinafter refers to coffee acidity as the brightness, sharpness or snappy taste a coffee possesses.) Another limitation of this approach is the introduction of ingredients that are not all natural, thereby adding artificial chemicals to the coffee that may have adverse health effects or drug interactions.

Another attempt to counter the detrimental effect of an acidic beverage on a person suffering from an acidic condition is described in U.S. Pat. No. 6,103,240 to Zhou on 15 Aug. 2000. Therein an herbal sweetening additive consisting of a triterpene glycoside composition and extract of licorice is described. One limitation of this reference is that the additive also alters the flavor of the beverage; specifically the additive sweetens the foodstuff to which it is added.

Another approach, described in U.S. Pat. No. 6,045,843 to Gurol on 4 Apr. 2000, modifies the processing of coffee beans to provide an acid-reduced whole bean coffee. Therein, a process includes the use of an aqueous solution including potassium hydroxide and polyethylene glycol applied to the whole bean coffee immediately after roasting. This process, however, is not well-suited for use by coffee drinkers in normal circumstances and relies, instead, on a specifically treated bean, which must be subsequently used to prepare a coffee drink. Moreover, the solution will alter the flavor of the coffee.

For people with peptic ulcer, many doctors who use herbal medicine use the deglycyrrhizinated form of licorice (DGL). In making DGL, the portion of licorice root that can increase blood pressure and cause water retention is removed, while the acid reducing, mucous-membrane-healing part of the root is retained, as discussed by Brogden, Speight, and Avery in their findings “Deglycyrrhizinated licorice: A report of its pharmacological properties and therapeutic efficacy” published in Drugs (1974) vol. 8, pgs. 330-9. DGL compares favorably to the popular drug cimetidine (Tagamet) for treatment of peptic ulcer according to D'imperio, Piccari, Sarti et al. in “Double blind trial in duodenal and gastric ulcers in Cimetidine and deglycyrrhizinized liquorice” published in Acta Gastro-Enterologica Belgica (1978) vol. 41 pgs. 427-34. And, after DGL and cimetidine were discontinued, Morgan, Pacsoo, and McAdam reported in their article “Maintenance therapy: a two year comparison between Caved-S and cimetidine treatment in the prevention of symptomatic gastric ulcer recurrence” published in Gut (1985) vol. 26 pgs. 599-602 fewer recurrences in the DGL group than in the cimetidine group.

Though not every trial has reported efficacy, for example as discussed by Bardhan, Cumberland, Dixon, and Holdsworth in their article “Clinical trial of deglycyrrhizinised liquorice in gastric ulcer” published in Gut (1978) vol. 19 pgs. 779-82, most studies find DGL to facilitate healing of peptic ulcer. A review of the DGL research shows that the studies not reporting efficacy used capsules, and the trials finding DGL to be helpful only when coming into contact with saliva according to Gaby in “Deglycyrrhizinated licorice treatment of peptic ulcer” published in Townsend Letter for Doctors (July 1988) pg. 306.

Thus, there remains a need for a beverage additive that reduces the acidity without overly affecting the flavor. Specifically, there is a need for a portable, easy-to-carry, easy-to-use, additive that can be added to coffee, enabling an acidic-condition sufferer to enjoy coffee, regardless of the source of the coffee, or the technique used to prepare the beverage. Additionally, such an additive should be all natural.

Further, a beverage additive that can readily be added to coffee that includes nutritional supplements would be highly desirable. For example, an additive that includes minerals, vitamins, and complex sugars to enhance both the taste and the nutritional value of the coffee is needed. Such a beverage additive specifically tailored to coffee must not negatively affect taste, but does augment the nutritional value of coffee is highly desirable.

Further, such an improvement to make coffee into a functional beverage should include substances that exploit scientific findings. These findings instruct that DGL may protect the stomach and duodenum by increasing production of mucin, a substance that protects the lining of these organs against stomach acid and other harmful substances according to the article by Goso, Ogata, Ishihara, and Hotta entitled “Effects of traditional herbal medicine on gastric mucin against ethanol-induced gastric injury in rats” published in Comp Biochem Physiol (1996) 113C: p. 17-21. According to laboratory research by Beil, Birkholz, and Sewing in “Effects of flavonoids on parietal cell acid secretion, gastric mucosal prostaglandin production and Helicobacter pylori growth,” published in Arzneimittelforschung (1995) vol. 45 pgs. 697-700, flavonoids in licorice may also inhibit growth of H. pylori.

### SUMMARY OF THE INVENTION

The various embodiments of the present invention provide a granulated solid additive that is ideally suited for dilution in coffee, and does not require alteration to the brewing process, the raw beans, the roasted beans, the grinding process, and the like. As such, the various embodiments of the present invention can be easily transported in a small packet, and carried by the user to be added to any drink the user desires. Further, the present invention in its many embodiments, is derived from all-natural, compounds that are generally recognized as safe (GRAS) under sections 201 (s) and 409 of the Federal Food, Drug, and Cosmetic Act.

In one embodiment of the present invention, an aqueous solution or solid mixture, reduces the effects on the stomach of acid in beverages when added. This solution of the present invention is an all-natural acid reducer comprising a special form of licorice called deglycyrhizinated form of licorice (DGL). The solution further may comprise ingredients including calcium, magnesium and fiber, but preferably only calcium and fiber, to help minimize acid production and prevent the painful effects of excessive stomach acid. Further, all of the solution's ingredients are generally recognized as safe by the United States government (or GRAS certified).

In another embodiment of the present invention, the beverage additive includes various mixtures of vitamins, minerals, natural caffeine, and other energy or nutritional enhancing components.

Benefits of the present invention include the reduction of the acidic effects of coffee and that it is fast-acting. Because it is immediately soluble in coffee, it works immediately to reduce the acidic effects of coffee.

The present invention further helps the body provide support against many types of acid related stomach disorders. Decreasing extra stomach acid can help relieve symptoms such as stomach pain, heartburn, difficulty swallowing, persistent cough, and trouble sleeping. It can also prevent serious acid damage to your digestive system.

In one embodiment of the present invention a specialized form of Licorice root called DGL, which has a long history of use for reducing acid, and soothing inflamed injured mucous membranes in the digestive tract. DGL may protect the stomach and duodenum by increasing production of mucin, a substance that protects the lining of these organs against stomach acid and other harmful substances. 1 According to laboratory research, flavonoids in licorice may also inhibit growth of H. pylori, a type of bacteria. Researchers believe that H. pylori is responsible for the majority of peptic ulcers.

In another embodiment, the present invention includes another form of fiber, a maltodextrin-like oligosaccharide, which is also known for its ability to promote healthy intestinal flora, which gets compromised by people with excessive stomach acid.

In a first preferred embodiment, the present invention consists of a composition of matter comprising: a mixture comprising about 20 percent deglycyrrhizinated licorice, about 45 percent Maltodextrin, about 20 percent Dolomite powder or Calcium Carbonate, and about 15 percent Inulin.

A first preferred method according to the present invention includes a method for altering a coffee-based beverage into a functional beverage comprising: providing an composition of matter according to any one of the preferred embodiments; providing a coffee-based beverage; and combining the composition with the coffee-based beverage.

A second preferred method according to the present invention includes a method for altering a coffee-based beverage by adding a composition of matter according to any one of the preferred embodiments; providing a coffee-based beverage; and combining the composition with the coffee-based beverage.

Another preferred embodiment of the present invention includes a functional beverage comprising: coffee; and any combination of the preferred embodiments, wherein the coffee and any combination of the compositions of matter create a mixture suitable for human consumption and having characteristics that improve health or provide other benefits to a human when consumed.

Additional additives to any of the preferred embodiments include, in any combination: about 5 percent yerba mate, at least about 15 percent guarana; about 30 percent glucosamine HCL with about 2 percent chondrotin sulfate; about 0.25 percent huperzine serrate; at least about 6 percent choline citrate; at least about 10 percent pantothenic acid (vitamin B5); about 2 percent ashawaganda root extract; about 15 percent Arginine; about 2 percent yohimbe extract; about 16 percent fructose; about 1.25 percent dandelion root; about 2 percent milk thistle root; about 40 percent Vitamin D; and/or about 15 percent kudzu root.

In other preferred embodiments, alone or in conjunction with previously disclosed additives, the present invention includes a composition of matter comprising about 600 mg fiber (or about 60% by weight) including prebiotic inulin for digestive heath benefits (similar to Bifidobacteria found in certain yogurt brands); and/or about 200 mg of calcium carbonate (or about 20% by weight) for improved bone health from improved calcium absorption.

### DESCRIPTION OF THE INVENTION

Possible embodiments will now be described with reference to the drawings and those skilled in the art will understand that alternative configurations and combinations of components may be substituted without subtracting from the invention. Also, in some figures certain components are omitted to more clearly illustrate the invention.

For people with peptic ulcer, many doctors who use herbal medicine use the deglycyrrhizinated form of licorice (DGL). In making DGL, the portion of licorice root that can increase blood pressure and cause water retention is removed, while the acid-reducing, mucous-membrane-healing part of the root is retained. In some reports, DGL has compared favorably to the popular drug cimetidine (Tagamet) for treatment of peptic ulcer.

The solution according to one embodiment of the present invention further includes common antacids such as calcium and magnesium. These common antacids have a long history of reducing stomach acid when consumed. They work by binding to excess acid produced by the stomach. This neutralizes the acid and decreases the acidity of the stomach content. The alkalizing properties of calcium and magnesium also help reduce the auto-toxication process.

The present invention also includes fiber extracted from inulin (chickory root) and a digestion resistant maltodextrin called Fibersol 2.

Inulin, a naturally occurring carbohydrate, is a dietary fiber and has the ability to activate the beneficial good bacteria in the digestive tract, which is critical to the health of people with excessive stomach acid. Inulin is used increasingly in foods because it has unusual nutritional characteristics. It ranges from completely bland to subtly sweet and can be used to replace sugar, fat, and flour. This is particularly advantageous because inulin contains a third to a quarter of the food energy of sugar or other carbohydrates and a sixth to a ninth of the food energy of fat. It also increases calcium absorption and possibly magnesium absorption, while promoting the growth of intestinal bacteria. Nutritionally, it is considered a form of soluble fiber and is sometimes seen as a prebiotic. The consumption of large quantities (particularly by sensitive or unaccustomed individuals) can lead to gas and bloating. Inulin has a minimal impact on blood sugar, and—unlike fructose—is not insulemic and does not raise triglycerides, making it generally considered suitable for diabetics and potentially helpful in managing blood sugar-related illnesses.

Fiber, such as Fibersol 2 brand maltodextrin, a digestion resistant maltodextrin, as a highly soluble fiber, helps absorb excessive acid. It also has been shown to increase the bioavailability of calcium, which can work favorably in reducing the autotoxication acid forming process. And, a maltodextrin, such as Fibersol 2 is very stable, has low viscosity, is transparent in solution and adds virtually no flavor to the finished product. Fibersol 2 brand maltodextrin is available from Matsutani Chemical America, 500 Park Blvd., Suite 1240, Itasca, Ill., 60143. Such a maltodextrin is produced from corn starch by pyrolysis and subsequent enzymatic treatment (similar to the process to manufacture conventional maltodextrins) to purposefully convert a portion of the normal alpha-1,4 glucose linkages to random 1,2-, 1,3-, and 1,4-alpha or beta linkages. The human digestive system effectively digests only alpha 1,4-linkages; therefore the other linkages render the molecules resistant to digestion.

Two prior art attempt at providing a low-acid coffee, U.S. Pat. No. 6,066,342 issued to Gurol et al, on 23 May 2000 and U.S. Pat. No. 5,853,787 to Gurol issued on 29 Dec. 1998, both assigned to Tamer International, Ltd (“Tamer”), teach methods and compositions to add to coffee before brewing resulting a coffee beverage having an acidity ranging from about pH 5.7 to about pH 6.1. The Tamer methods teach adding the acid-reducing powder, granules, or liquid to whole beans prior to the brewing of the beverage, or adding to the ground coffee prior to brewing. Alternatively, the additive may be included in a creamer, provided on a laced filter, or be added to coffee. Further, numerous of the ingredients taught by these references are artificial, including magnesium hydroxide, and such ingredients are disfavored by today's coffee-drinking population.

In contrast, the present invention reduces the affect of acid on the human body, and does not (necessarily) alter the acidity of the beverage itself. And, as such, does not alter the flavor of the coffee or coffee-based beverage and is specifically formulated not to interfere with the taste of the coffee drink. The present invention includes both antacid and digestive health benefits, including the use of DGL, a natural substance known to compare favorably with Tagement, fiber, a prebiotic known to improve digestive health and regularity with prolonged use. The present invention, in certain preferred embodiments includes only four, all-natural ingredients, which is more economical to produce and meets the demand's of today's consumers that insist on healthful products using natural ingredients.

Other differences of the present invention include the use of all-natural and/or organic ingredients not contemplated by the prior-art as will be appreciated by those skilled in the art when the following exemplary embodiments are discussed in detail, below. Further, by reducing the effects of acidity, and not the acidity of the coffee drink itself, the desired taste, texture and brightness of coffee remains.

Additional benefits of the present invention not taught or contemplated in the prior art include improved digestive health through the incorporation of prebiotics (similar to the prebiotics in yogurt, cheese and other foods), such as inulin. However, unlike food such as yogurt and cheese, the present invention is more easily carried by the user and offers significantly less calories and fat content than yogurt, cheese, and similar dairy products.

Other benefits of the present invention include transforming traditional coffee or any beverage containing coffee into a functional beverage, or adding nutritional supplements to remedy ailments or conditions such as a hang-over or stress, or improving bone health.

Additionally, throughout this document, the present invention references coffee, coffee drinks, and coffee-based beverages synonymously. A coffee-based beverage is any drink for humans that contains coffee including, but not limited to brewed coffee, French press, cold-pressed, steamed coffee, drip coffee, percolated coffee, iced coffee, expresso, expresso drinks, lattes, mochas, macchiato, cappuccino, specialty coffee drinks, coffee with syrups, coffee with sweeteners, coffee with alcohol, and any combination thereof.

### EXAMPLE 1

One embodiment of the present invention consists of an additive for reducing the affects of acidity on a human stomach and generally to improve digestive health. The essential ingredients include a mixture comprising, preferably about 20 percent, deglycyrhizinated licorice (DGL) from about 5 percent to about 60 percent volume by weight.

The mixture further comprises about 45 percent Fibersol-2 produced by Archer Daniels Midland Company (ADM) of Decatur, Ill., USA and is digestion resistant maltodextrin that is an innovative, highly soluble fiber. Usage levels of Maltodextrin range from about 15 percent to about 70 percent volume by weight.

The mixture further comprises about 20 percent Dolomite powder or, preferably, Calcium Carbonate, a natural source mineral that contains calcium in the form of a carbonate. Usage levels of calcium carbonate range from about 5 percent to about 70 percent volume by weight in the formulation of this exemplary embodiment.

The mixture further comprises about 15 percent inulin, also known as Oliggo-Fiber available from Cargill Inc., of Minneapolis, Minn., USA. Inulin is a line of oligosaccharide products belonging to the group of non-digestible carbohydrates called fructans and naturally occurs as fructan extracted from chicory root. Usage levels of inulin range from about 5 percent to about 40 percent volume by weight.

EXAMPLE 1
Javacid Original - Digestive Health

| | Preferred volume | |
|---|---|---|
| Blending Material | % by weight | Range % by weight |
| DGL | 20 | 5-60 |
| Fibersol 2 | 45 | 15-70 |
| Calcium Carbonate or Dolomite | 20 | 5-70 |
| Inulin | 15 | 5-40 |

### EXAMPLE 2

In a second embodiment of the present invention, an additive for reducing the affects of acidity, such as Example 1, is further enhanced to provide increased benefit for the beverage drinker and includes immune-system specific additives. In addition to the formulation of the mixture in Example 1, the following additional ingredients are included:

About 5 percent Echinacea angustafolia, a specific species of the natural herbal plant Echinacea having the highest concentration of immune stimulating phenols called echinacosides. Usage levels range from about 3 percent to about 20 percent volume by weight.

In addition, the mixture further comprises about at least 4 percent zinc gluconate powder. Zinc has been shown in numerous scientific studies to reduce the duration of the common cold and should be included in a usage range from about 3 percent to about 20 percent volume by weight.

EXAMPLE 2
Javacid Immune

| | Preferred volume | |
|---|---|---|
| Blending Material | % by weight | Range % by weight |
| DGL | 18 | 5-60 |
| Fibersol 2 | 41 | 15-70 |
| Calcium Carbonate or Dolomite | 18 | 5-70 |
| Inulin | 14 | 5-40 |
| Echinacea Angustafolia (4% | 5 | 1-15 |
| Echinacosides) | | |
| Zinc Gluconate Powder | 4 | 1-15 |

### EXAMPLE 3

In a third embodiment of the present invention, an additive for reducing the affects of acidity, such as Example 1, is further enhanced to provide increased benefit for the beverage drinker and includes energy-boosting ingredients. The mixture of Example 1 further comprises about 5 percent Yerba mate, a natural herb that extends the duration of caffeine in a product and should be included in a range from about 3 percent to about 7 percent volume by weight in the formulation of this embodiment.

And, this mixture further comprises about at least 15 percent of natural caffeine from coffee sources and should be used in the range of about 3 percent to about 25 percent volume by weight in this exemplary formulation.

EXAMPLE 3
Javacid Energy

| | Preferred volume | |
|---|---|---|
| Blending Material | % by weight | Range % by weight |
| DGL | 17 | 5-60 |
| Fibersol 2 | 37 | 15-70 |
| Calcium Carbonate or Dolomite | 17 | 5-70 |
| Inulin | 12 | 5-40 |
| Yerba Mate | 4 | 1-15 |
| Guarana (22% Caffeine) | 13 | 5-35 |

### EXAMPLE 4

In a fourth embodiment of the present invention, an additive for reducing the affects of acidity, such as Example 1, is further enhanced to provide increased benefit for the beverage drinker and includes additives to enhance movement of the joints. Accordingly, the mixture of Example 1 further comprises about 30 percent glucosamine hydrochloride, a natural dietary supplement ingredient that can stimulate specific components of a user's cartilage to thrive and reproduce. It should be used in a range from about 20 percent to about 60 percent volume by weight in this exemplary formulation.

In addition, this mixture further comprises about 20 percent chondrotin sulfate, a compound that can block the enzymes that destroy crucial cartilage tissue, ranging from about 10 percent to about 40 percent volume by weight in this embodiment.

EXAMPLE 4
Javacid Flex Comfort

| | Preferred volume | |
|---|---|---|
| Blending Material | % by weight | Range % by weight |
| DGL | 13 | 5-60 |
| Fibersol 2 | 30 | 15-70 |
| Calcium Carbonate or Dolomite | 13 | 5-70 |
| Inulin | 10 | 5-40 |
| Glucosamine HCL | 20 | 5-40 |
| Chondrotin Sulfate | 13 | 3-25 |

### EXAMPLE 5

In a fifth embodiment of the present invention, an additive for reducing the affects of acidity, such as Example 1, is further enhanced to provide increased benefit for the beverage drinker and includes additives to enhance memory. Accordingly, the mixture of Example 1 further comprises about 0.25 percent huperzine serrate, a powerful compound extracted from the club moss Huperzia Serrate. It is a popular mind-supporting supplement that promotes enhanced brain function. Huperzine-A supports the nervous system's role in transmitting acetylcholine information from cell to cell, and promotes stronger nervous connections for healthy cognitive function and memory. In this embodiment, it is used in a range from about 0.15 percent to about 0.50 percent.

In addition, this mixture further comprises about at least 6 percent choline citrate, a nutritional building-block for the neurotransmitter acetylcholine. Usage levels range from about 5 percent to about 8 percent volume by weight in this embodiment.

EXAMPLE 5
Javacid Memory

| | Preferred volume | |
|---|---|---|
| Blending Material | % by weight | Range % by weight |
| DGL | 19 | 5-60 |
| Fibersol 2 | 42 | 15-70 |
| Calcium Carbonate or Dolomite | 19 | 5-70 |
| Inulin | 14 | 5-40 |
| Huperzine Serrate (1% Hup A) | 0.0 | 0.01-5 |
| Choline Citrate | 6 | 1-20 |

### EXAMPLE 6

In a sixth embodiment of the present invention, an additive for reducing the affects of acidity, such as Example 1, is further enhanced to provide increased benefit for the beverage drinker and includes additives to enhance relaxation and de-stressing. Accordingly, the mixture of Example 1 further comprises about at least 1.0 percent pantothenic acid (vitamin B5) and can be used in a range from about 0.5 percent to about 3.0 percent volume by weight.

In addition, this mixture further comprises about at least 10 percent ashawaganda root extract, is a natural herbal compound that is used as an adaptogen and stress reducer. Usage levels range from about 5.0 percent to about 30.0 percent volume by weight in this formulation.

EXAMPLE 6
Javacid De-Stress

| | Preferred volume | |
|---|---|---|
| Blending Material | % by weight | Range % by weight |
| DGL | 18 | 5-60 |
| Fibersol 2 | 40 | 15-70 |
| Calcium Carbonate or Dolomite | 18 | 5-70 |
| Inulin | 13 | 5-40 |
| Pantothenic Acid (Vitamin B5) | 1 | 1-5 |
| Ashawaganda Root Extract | 9 | 1-30 |

### EXAMPLE 7

In a seventh embodiment of the present invention, an additive for reducing the affects of acidity, such as Example 1, is further enhanced to provide increased benefit for the beverage drinker and includes additives to enhance libido. Accordingly, the mixture of Example 1 further comprises about at least 15 percent Arginine, a precursor to the naturally containing compound nitric oxide in the human body. Usage levels of Arginine in the form of hydrochloride, ethyl ester or free-base can be used in a range from about 10 percent to about 30 percent volume by weight in this formulation.

In addition, this mixture further comprises about at least 10 percent yohimbe extract in the form of powder, standardized extract or hydrochloride. Usage levels range from about 2.0 percent to about 30.0 percent volume by weight in this formulation.

EXAMPLE 7
Javacid Libidio

| | Preferred volume | |
|---|---|---|
| Blending Material | % by weight | Range % by weight |
| DGL | 16 | 5-60 |
| Fibersol 2 | 36 | 15-70 |
| Calcium Carbonate or | 16 | 5-70 |
| Dolomite | | |
| Inulin | 12 | 5-40 |
| Arginine | 12 | 1-30 |
| Yohimbe extract | 8 | 1-25 |
| (2% Yohimbine) | | |

### EXAMPLE 8

In an eighth embodiment of the present invention, an additive for reducing the affects of acidity, such as Example 1, is further enhanced to provide increased benefit for the beverage drinker and includes additives to reduce the affects of a hangover. Accordingly, the mixture of Example 1 further comprises about 16 percent fructose, a natural sugar from fruit that speeds up the metabolism process when the liver detoxifies alcohol. Usage levels range from about 1 percent to about 25 percent volume by weight in this embodiment.

In addition, this mixture further comprises about at least 12.5 percent dandelion root, which can also assist the liver to detoxify harmful substances such as alcohol. Usage levels can range from about 10 percent to about 30.0 percent volume by weight in this formulation.

And, the mixture further comprises other herbal compounds that can speed up the rate at which alcohol is metabolized in the liver utilizing about 10.0 percent milk thistle root in a usage level ranging from about 5.0 percent to about 20.0 percent and from about 15 percent kudzu root in a usage level of from about 5.0 percent to 20.0 percent (volumes by weight).

EXAMPLE 8
Javacid Hangover

| | Preferred volume | |
|---|---|---|
| Blending Material | % by weight | Range % by weight |
| DGL | 7 | 5-60 |
| Fibersol 2 | 15 | 15-70 |
| Calcium Carbonate or | 7 | 5-70 |
| Dolomite | | |
| Inulin | 5 | 5-40 |
| Fructose | 54 | 20-80 |
| Dandelion Root | 4 | 1-15 |
| Milk Thistle Root | 3 | 1-10 |
| (80% Silymarin) | | |
| Kudzu Root | 5 | 1-15 |

### EXAMPLE 9

In a ninth preferred embodiment of the present invention, a system of additives for food and includes a collection of beverage additives targeted to improve general bone health and that selectively adds nutrients, minerals, sugars, vitamins, and other all-natural components to a coffee-based beverage.

EXAMPLE 9
Javacid Bone Health

| | Preferred volume | |
|---|---|---|
| Blending Material | % by weight | Range % by weight |
| Calcium Carbonate or Dolomite | 40 | 5-70 |
| Inulin | 20 | 5-40 |
| Vitamin D | 40 | 20-80 |

The present invention contemplates a number of particular coffee enhancements that enables the beverage drinker to custom tailor their favorite coffee drink to include additives that assist in calming stress, adding energy, reducing swelling of the joints, increasing mental functions, the libido, hangover relief, or multivitamin consumption. Other additives are contemplated include cold-relief, head-relief, muscle relief, nausea relieve, added fibers, etc. The various formulations of the present invention will work equally well with other beverages including tea, for example.

The present invention, in many of the presented preferred embodiments, is generally contemplated as a stand-alone, pre-measured additive to a coffee-based beverage: However, it should also be understood that the present invention will work equally well in bulk quantities, and may be added to known coffee enhancing substances including, but not limited to, granular, powder or liquid sweeteners, creamers, syrups, flavorings, milk, and the like, for instance.

Although the invention has been particularly shown and described with reference to certain embodiments, it will be understood by those skilled in the art that various changes in form and detail may be made without departing from the spirit and scope of the invention. And, although claims are not required, I claim at least:

I claim:

1. A method for reducing the effects of acidic beverages consumed by and improving the digestive health of a human, the method comprising:
providing a coffee-based beverage; and
adding to the coffee-based beverage a composition comprising a mixture comprising from 5 percent to 60 percent deglycyrhizinated licorice, from 15 percent to 50 percent maltodextrin, and from 5 percent to 40 percent inulin.

2. The method of claim 1 further comprising:
providing a pre-determined amount of the composition in a disposable container.

3. The method of claim 1 wherein the composition comprises:
20.0 percent deglycyrrhizinated licorice
45.0 percent Maltodextrin, and
15.0 percent Inulin.

4. The method of claim 1 wherein the composition comprises:
20.0 percent Calcium Carbonate.

5. The method of claim 1 wherein the composition comprises:
Echinacea angustafolia, and zinc gluconate powder.

6. The method of claim 1 wherein the composition comprises:
yerba mate, and guarana.

7. The method of claim 1 wherein the composition comprises:
glucosamine HCL; and chondrotin sulfate.

8. The method of claim 1 wherein the composition comprises:
huperzine serrate and choline citrate.

9. The method of claim 1 wherein the composition comprises:
pantothenic acid (vitamin B5), and
ashawaganda root extract.

10. The method of claim 1 wherein the composition comprises:
Arginine, and
yohimbe extract.

11. The method of claim 1 wherein the composition comprises:
fructose,
dandelion root,
milk thistle root, and
kudzu root.

12. A method for transforming a coffee-based beverage into a functional beverage, the method comprising:
providing the coffee-based beverage;
providing a pre-determined amount of a composition wherein the composition comprises a mixture comprising from 5 percent to 60 percent deglycyrhizinated licorice, from 15 percent to 50 percent maltodextrin, and from 5 percent to 40 percent inulin; and
adding the mixture to the beverage.

13. The method of claim 12 wherein the mixture comprises:
Echinacea angustafolia, and zinc gluconate powder.

14. The method of claim 12 wherein the mixture comprises:
yerba mate, and guarana.

15. The method of claim 12 wherein the mixture comprises:
glucosamine HCL; and chondrotin sulfate.

16. The method of claim 12 wherein the mixture comprises:
huperzine serrate and choline citrate.

17. The method of claim 12 wherein the mixture comprises:
pantothenic acid (vitamin B5), and
ashawaganda root extract.

18. The method of claim 12 wherein the mixture comprises:
arginine, and
yohimbe extract.

19. The method of claim 12 wherein the mixture comprises:
Vitamin D.

20. The method of claim 12 wherein the mixture consists of any combination of the following: dandelion root, milk thistle root, and kudzu root, sweetner, creamer, milk, cream, syrup, sugar, and flavorings.
